(19) United States
(12) Patent Application Publication
(10) Pub. No.: US 2026/0000437 A1
(43) Pub. Date: Jan. 1, 2026

(54) REPAIR APPARATUS FOR REPAIRING BONE DEFECTS

(71) Applicant: University of Central Oklahoma, Edmond, OK (US)

(72) Inventors: Morshed Khandaker, Edmond, OK (US); Amgad Haleem Amen, Belmont, MA (US)

(21) Appl. No.: 19/253,319

(22) Filed: Jun. 27, 2025

Related U.S. Application Data
(60) Provisional application No. 63/665,905, filed on Jun. 28, 2024.
(60) Provisional application No. 63/665,846, filed on Jun. 28, 2024.

(51) Int. Cl. A61B 17/72; A61B 17/00; A61B 17/84; B33Y 10/00

(52) U.S. Cl. CPC A61B 17/72; A61B 17/84; A61B 2017/00004; A61B 2017/00933; B33Y 10/00

(57) ABSTRACT

A repair apparatus for repairing bone defects includes a bioabsorbable scaffold having first and second ends that spans a space between spaced apart first and second bone fragments. The first end of the scaffold envelops an end of the first bone fragment and the second end of the scaffold envelops an end of the second bone fragment.

### CROSS REFERENCE TO RELATED APPLICATIONS

This application claims priority from and the benefit of U.S. Provisional Application 63/665,905 filed Jun. 28, 2024, and U.S. Provisional Application 63/665,846 filed Jun. 28, 2024, both of which are incorporated by reference.

### STATEMENT REGARDING FEDERALLY SPONSORED RESEARCH OR DEVELOPMENT

This invention was made with government support under P20GM103447 awarded by the National Institutes of Health. The government has certain rights in the invention.

### BACKGROUND

A critical size bone defect is generally defined as the smallest size intra-osseous wound in a particular bone and species of animal that will not heal spontaneously or as a defect that shows less than 10% bone regeneration during the lifetime of the animal. A defect can also be characterized as ‘critical size’ when its length deficiency exceeds two to three times its diameter. The clinical treatment of critical-size bone defects remains a challenge worldwide due to several problems associated with autologous bone grafting with metallic intramedullary nail systems, which is the current clinical gold standard for humans.

While bone tissue engineering holds promise for the regeneration of segmental bone defects, challenges still exist with regard to effective scaffold design and translational animal studies. Developing bioresorbable bone substitutes that have strength characteristics similar to cortical bone strength and have osteoinductive properties may provide an effective method to treat the clinical large segmental bone defects. Using a bioabsorbable scaffold and intramedullary nail that can be absorbable may offer many advantages over the autologous bone substitute solutions, including reduced recovery time, load bearing during the recovery process, and no need for future surgical procedures to remove the implants due to their bioabsorbable property.

Current segmental bone loss treatment options include distraction osteogenesis, induced membrane, and vascularized fibular transplantation with an interlocked intramedullary nail, made with metal as depicted in FIG. 1B. Each of these options has its advantages, but they also have significant drawbacks and complications. In an effort to improve and hasten healing in human segmental defects, new and emerging technologies such as a tissue-engineered bone graft has been developed, which can be assembled with the IM nail in the bone defect area (FIG. 1C). There is currently no standardized treatment method for critical-size segmental long bone defects for humans. Current solutions remain unsatisfactory, requiring multiple complex surgeries, with a high risk of infection, and often leaving patients with lifelong difficulties. A new solution is needed which promotes bone union while allowing weight bearing during the healing process to aid long-term results and which requires only a single minimally invasive surgery.

Disclosed herein are bone substitutes that are bioresorbable and have osteoconductive and osteoinductive properties to treat clinical large critical-size bone defects. Using a metallic intramedullary nail, and a bioabsorbable scaffold circumferentially covered with biologic nanofiber, and screws fixing the scaffold and the nail to the bone segments of the critical size bone may offer many advantages over the metallic intramedullary nail with autologous bone substitutes solutions, including reduced recovery time due to the bioabsorbable property with biodegradation, allowing creep substitution with regenerative bone tissue.

### SUMMARY

Aspects of this disclosure are directed to the development of bone substitutes that are bioresorbable, osteoconductive, and have osteoinductive properties for treatment of clinical large critical-size bone defects. Disclosed herein are an apparatus and method for using an intramedullary nail and a bioabsorbable scaffold. The bioabsorbable scaffold may comprise first and second, or inner and outer scaffold portions. In one implementation, the outer scaffold portion comprises biologic nanofiber that circumferentially covers the inner scaffold portion. Screws may be used to fix the intramedullary nail and the scaffold to the bone. Such an apparatus and method may reduce recovery time due to the bioabsorbable property with biodegradation, allowing creep substitution with regenerative bone tissue.

The bioabsorbable scaffold in one aspect may span the distance between the ends of bone fragments in a critical size defect and may extend over and envelop the ends of the separated bone fragments. The intramedullary nail may be metallic or may be made from bioabsorbable materials (e.g., ceramics, polymer, composites or other bioabsorbable material). In one embodiment, the bioabsorbable scaffold may comprise a 3D printed bioabsorbable scaffold with electrospun nanofibers, which may be an electrospun biological nanofiber mesh wrapped thereabout. The bioabsorbable scaffold may be secured with screws extending into the bone fragments and an intramedullary nail. The porous bioabsorbable scaffolds and nanofiber mesh may act as a bioartificial bone substitute and may therefore constitute a segmental bone graft. The 3D-printed tissue-engineered segmental bone graft and corresponding metallic or non-metallic interlocking nail system, specific to lesion size, site, and geometry disclosed herein, may allow early weight bearing in addition to other improvements over currently available techniques.

### BRIEF DESCRIPTION OF THE FIGURES

Various aspects of the present disclosure are illustrated in the following detailed description and accompanying figures.

FIG. 1A is a depiction of a tibia bone.

FIG. 1B is a depiction of a tibia bone with a critical bone defect, along with an intramedullary nail extending into the bone fragments and secured by screws.

FIG. 1C is a depiction of a tibia bone with a critical bone defect, along with an intramedullary nail extending into the bone fragments and secured by screws. A scaffold is positioned between the bone fragments to act as a bone graft.

FIG. 2 is an elevation view of a repair apparatus of the current disclosure.

FIG. 3 is a cross section of the repair apparatus of FIG. 2.

FIG. 4 is an elevation view of an additional embodiment of a repair apparatus of the current disclosure.

FIG. 5 is a cross section of the embodiment of FIG. 4.

FIG. 6 is an elevation view of an additional embodiment of a repair apparatus of the current disclosure.

FIG. 7 is a cross section of the embodiment of FIG. 6.

FIG. 8 is an elevation view of an additional embodiment of a repair apparatus of the current disclosure.

FIG. 9 is a cross section of the embodiment of FIG. 8.

FIG. 10 is a cross section of an additional embodiment of a repair apparatus.

### DETAILED DESCRIPTION

Aspects of this disclosure are described in greater detail below.

As used herein, the terms “comprises,” “comprising;” or any other variation thereof are intended to cover a non-exclusive inclusion, such that a process, method, composition, article, or apparatus that comprises a list of elements does not include only those elements but may include other elements not expressly listed or inherent to such process, method, composition, article, or apparatus. The term “exemplary” is used in the sense of “example” rather than “ideal.”

As used herein, the singular forms “a,” “an,” and “the” include plural reference unless the context dictates otherwise. The terms “approximately” and “about” refer to being nearly the same as a referenced number or value. As used herein, the terms “approximately” and “about” should be understood to encompass ±5% of a specified amount or value.

The present disclosure addresses the needs described above by providing a repair apparatus and method to repair critical bone defects. The apparatus in one embodiment comprises a 3D-printed tissue-engineered segmental bone graft and corresponding metallic or non-metallic interlocking nail system, specific to lesion size, site, and geometry. The repair apparatus may allow sufficient early weight bearing, and may have bioabsorbable characteristics and cellular elements that aid in bone growth.

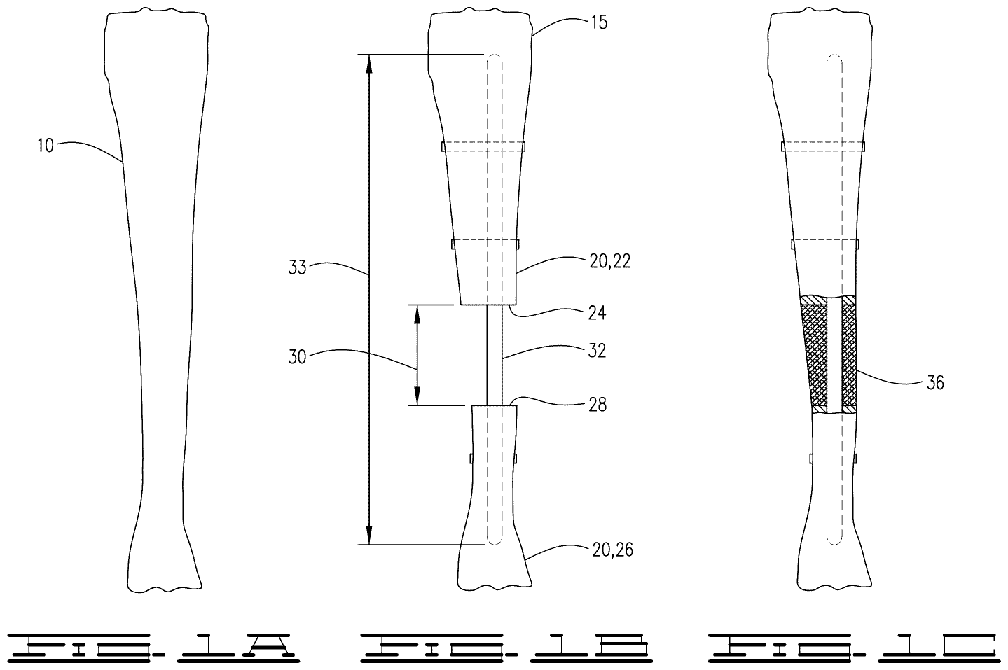

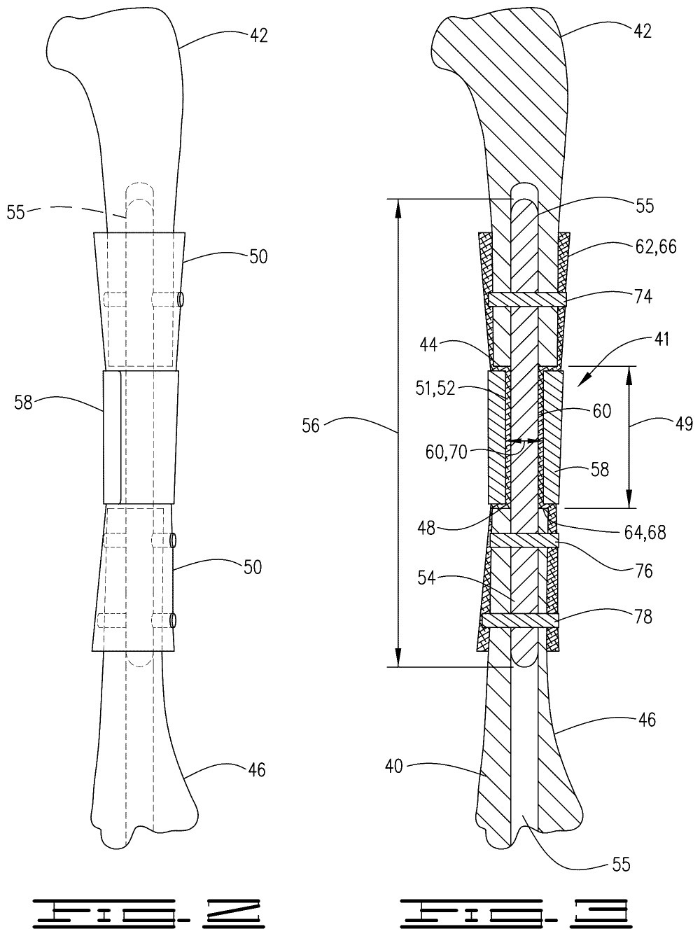

Referring now to the figures, FIG. 1A depicts a tibia 10 and FIGS. 1B and 1C depict a tibia 15 with a critical bone defect addressed by a prior art repair. The critical bone defect comprises a fracture resulting in bone fragments 20, which comprise a first, or upper bone fragment 22 with an end 24 and a second, or lower bone fragment 26 with an end 28. Bone fragments 22 and 26 have space 30 therebetween. An intramedullary metal nail 32 with a length 33 extends into the upper and lower bone fragments 22 and 26. A 3D printed scaffold bone graft 36 is positioned between the ends 24 and 28 of bone fragments 22 and 26. Upper and lower bone fragments 22 and 26 will have lengthwise holes created therein to receive the intramedullary nail 32.

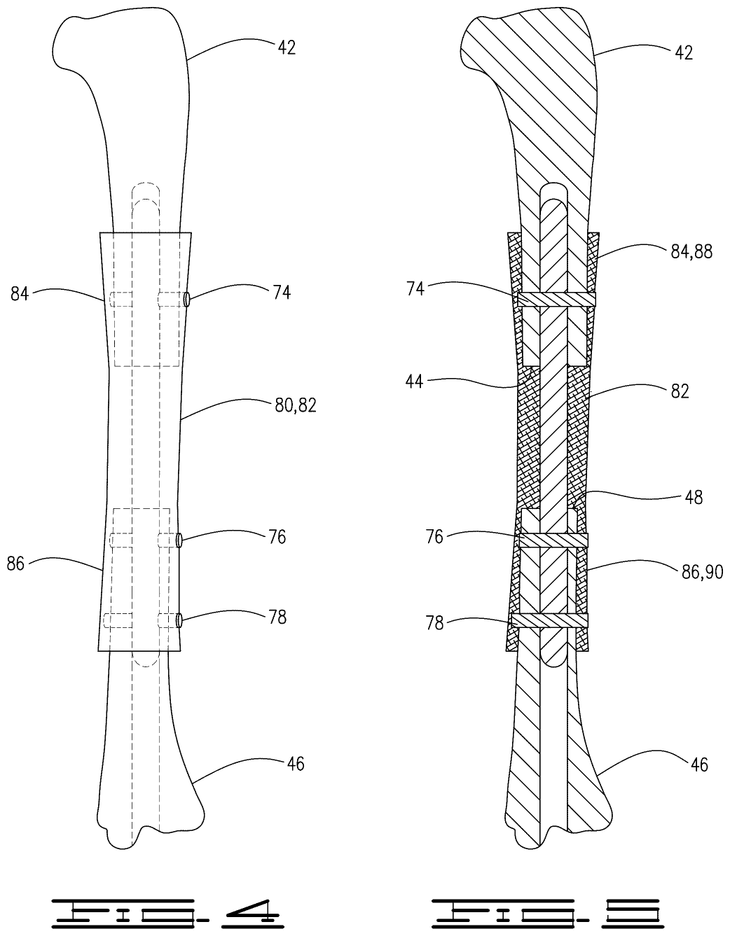

Referring now to FIGS. 2 and 3, tibia 40 with a bone defect 41 is shown. Bone defect 41 comprises bone fragments 42 and 46 with a space 49 therebetween. First, or upper bone fragment 42 has an end 44 and second, or lower bone fragment 46 has an end 48. A repair apparatus 50 is shown connecting bone fragments 42 and 46. Repair apparatus 50 may comprise a scaffold 51, that includes an inner portion or inner scaffold portion 52, which may be a 3D printed tissue-engineered critical-size bone scaffold, specific to the lesion size, site, and geometry. A scaffold, as is well known in the art and context of bone repair, is a structure designed to bridge gaps in damaged bones, and to support the growth of new bone. Repair apparatus 50 may further comprise an intramedullary nail 54 with a length 56. Intramedullary nail 54 may be metal or non-metal and may be bioabsorbable or non-bioabsorbable. An opening 55 in which intramedullary nail 54 is received may in one implementation extend from a lower end of lower bone fragment 46 through bone fragment 46 and into bone fragment 42. In other implementations, the opening 55 may be drilled from ends 44 and 48 of bone fragments 42 and 46 respectively and will not be through holes. In other words, opening 55 in some implementations will not extend completely through either of bone fragments 42 and 46. In the embodiment of FIG. 3, scaffold 51 of repair apparatus 50 comprises an outer portion, or outer scaffold portion 58 which may comprise a nanofiber mesh 58 circumferentially disposed about a portion of inner scaffold portion 52. Inner and outer scaffold portions 52 and 58 may be referred to as first and second scaffold portions 52 and 58, respectively.

Outer scaffold portion 58 may comprise a nano fiber mesh which may be an electrospun nanofiber mesh. The nanofiber mesh may be produced by methods known in the art. One method involves dissolving PCL in acetone to create a solution, and feeding the solution through a single-axis, one-inch discharge metallic needle (Model #BX 25). An electrospinning machine includes a drum collector, which can be operated by speed-controlled direct current (DC) motors. A high voltage of 9 kV, generated by a high precision and high voltage power supply AC-DC conversion MAX output-20 KV 0.5 mA (Analog Technologies, Inc., San Jose, CA, USA), is applied to the syringe needle, creating an electrically charged jet in the PCL solution. The jet is directed toward the drum collector, located approximately 5 cm away from the needle at room temperature and relative humidity of 30-40%, to form a stream of synthetic polymer fibers. The parameters, such as the rotation speed of the drum, the needle-drum distance, and the fiber deposition rate, are set to optimize fiber mat formation. The solution feeding rate can be set as desired, and in one embodiment may be, for example, 0.025 mL/minute. The drum can be of different sizes and in one embodiment 40 mm. The foregoing is exemplary, and it is understood that the electrospun nanofiber mesh can be made using other equipment and parameters.

Inner scaffold portion 52 may be a 3D-printed Polycaprolactone (PCL) scaffold. In one embodiment, the PCL scaffold portion 52 can be soaked with alginate-hydroxyapatite hydrogel or other bone marrow mesenchymal stem cells (BMSC) biocompatible gel to aid BMSC cell growth in critical-size bone defects. The PCL scaffold portion may comprise polycaprolactone-hydroxyapatite (PCL-HA). The produced 3D-printed PCL intramedullary nail system (repair apparatus 50) can osseointegrate and slowly degrade and absorb with bone.

In the embodiment of FIG. 2, the porous inner scaffold portion 52 may have a central portion 60 with first, or proximate and second, or distal ends 62 and 64 respectively disposed about the intramedullary nail 54. Central portion 60 has an outer diameter 70 that is smaller than an outer dimension of the bone fragments 42 and 46. Inner scaffold portion 52 has a first, or proximate socket 66 at proximate end 62 and a second, or distal socket 68 at distal end 64. Socket 66 will extend over the end 44 of bone fragment 42, and will envelop, or encapsulate end 44. Socket 68 will extend over the end 48 of bone fragment 46, and will envelop, or encapsulate end 48. Sockets 66 and 68 are patient specific, in that the shapes are designed specifically for the bone fragments of the critical bone defect to be repaired. The outer diameter, or outer dimension 70 of central portion 60 is smaller than the outer diameter of sockets 66 and 68. Nanofiber mesh 58 may be circumferentially wrapped around central portion 60 of scaffold 52 to produce a nanofiber mesh incorporated PCL scaffold. The nanofiber mesh may be an electrospun nanofiber mesh produced by methods known in the art, such as that described above. The nanofiber mesh can be immobilized with bio molecules such as, for example, bone growth protein, peptide, and growth factors. The nanofiber mesh serves as a carrier for BMSC, bone growth factors, antimicrobial agents, and heal-tracking molecules, promoting natural bone regeneration, resisting infection and monitoring healing.

Biomolecules can be immobilized onto nanofibers in a variety of ways including for example, encapsulation, adsorption, and covalent bonding. Encapsulated biomolecules are not attached to the nanofiber surface but are entrapped in the polymer network. The biomolecule is added to the spinning solution and becomes immobilized in the polymer matrix during the electrospinning process. In biomolecule immobilization by adsorption, the biomolecule and the nanofiber substrate are placed in solution for a fixed amount of time and then rinsed with buffer solution to remove any unabsorbed biomolecules. Another method comprises covalently binding the biomolecule to the nanofiber surface. Stable complexes are formed between the functional groups of the substrate and the functional groups of the biomolecule. The binding biomolecule may be bound onto the nanofiber substrate by direct reaction onto the substrate or activation of the surface through the use of crosslinkers.

A plurality of screws may be used to affix the scaffold 51 and the intramedullary nail to the bone fragments 42 and 46. At least one screw may be used above and below the critical bone defect. In the described embodiment, first and second screws 74 and 76 are positioned to pass through the scaffold 51 and bone fragments 42 and 46 respectively, and through intramedullary nail 54. An additional screw 78 is positioned below screw 76 and extends through bone fragment 46 and socket 68 of scaffold 51 into intramedullary nail 54. The screws may be metal screws or may be 3D printed biocompatible screws. The method of repair may comprise 3D printing the inner scaffold portion 52, depositing the nanofiber mesh onto the 3D printed inner scaffold portion 52, positioning the assembled scaffold 51 in the space between bone fragments 42 and 46, placing the ends of the bone fragments 42 and 46 in first and second sockets 66 and 68. A metal intramedullary nail 54 may then be inserted through the axial opening from the bottom of the lower bone fragment 46, through the opening in the scaffold 51 positioned in the space between the bone fragments 42 and 46 and into the axial opening in upper bone fragment 42. In the case where a non-metal intramedullary nail is used, the axial openings in the upper and lower bone fragments do not need to be through holes. See for example the embodiment of FIGS. 6 and 8. A flexible bioabsorbable nail may be inserted though the inner scaffold portion 52 and the nail flexed to insert into the spaced apart ends of the upper and lower bone fragments 42 and 46.

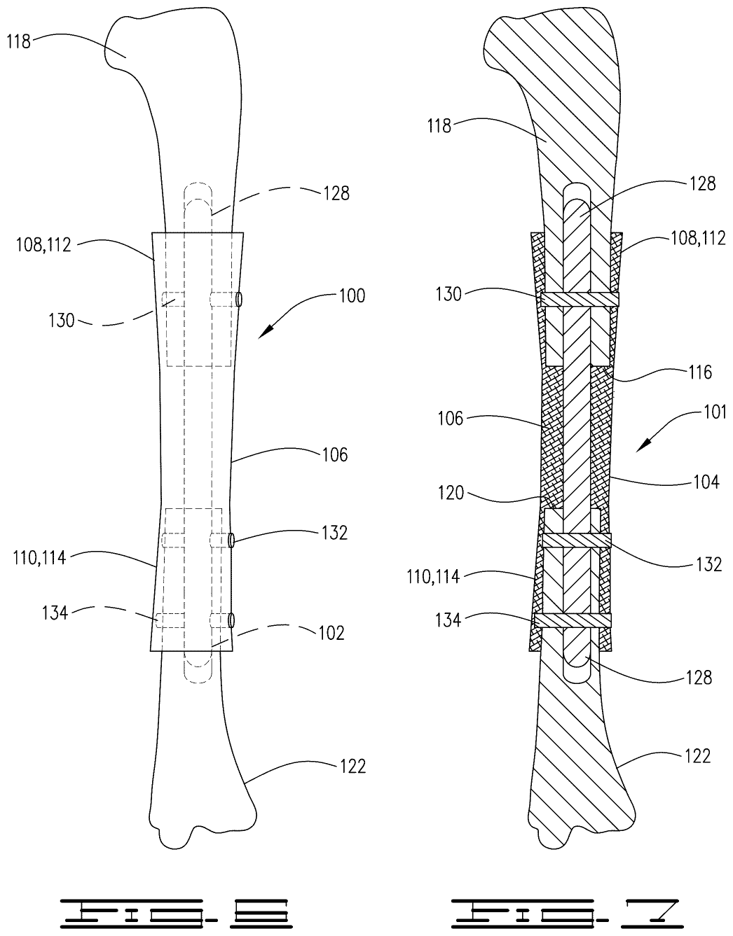

An additional embodiment of a repair apparatus 80 (interlocking intramedullary nail system) is shown in FIGS. 4 and 5. Repair apparatus 80 comprises a porous scaffold 82, that like scaffold 51 may be a 3D-printed PCL scaffold. Repair apparatus 80 is like repair apparatus 50, except that it does not include a nanofiber mesh, and the PCL scaffold 82 fills the space 49 between ends 44 and 48 of upper and lower bone fragments 42 and 46. Scaffold 82 is a single component scaffold 82. Scaffold 82, like scaffold 51 has sockets 88 and 90 at the ends 84 and 86 thereof. Sockets 88 and 90 extend over ends 44 and 48 of upper and lower bone fragments 42 and 46 respectively, and envelop, or encapsulate ends 44 and 48. At least one screw may be used above and below the critical bone defect to secure the scaffold. In the described embodiment, first and second screws 74 and 76 are positioned to pass through the scaffold 82 and bone fragments 42 and 46 respectively, and through intramedullary nail 54. An additional screw 78 may be positioned below screw 76 and extends through socket 90 of scaffold 82 and bone fragment 46 into intramedullary nail 54. The method of repair may comprise 3D printing the scaffold 82 and inserting a metal intramedullary nail through the axial opening from the bottom of the lower bone fragment 46, through the scaffold 82 positioned in the space between the bone fragments 42 and 46 and into the axial opening in upper bone fragment 42.

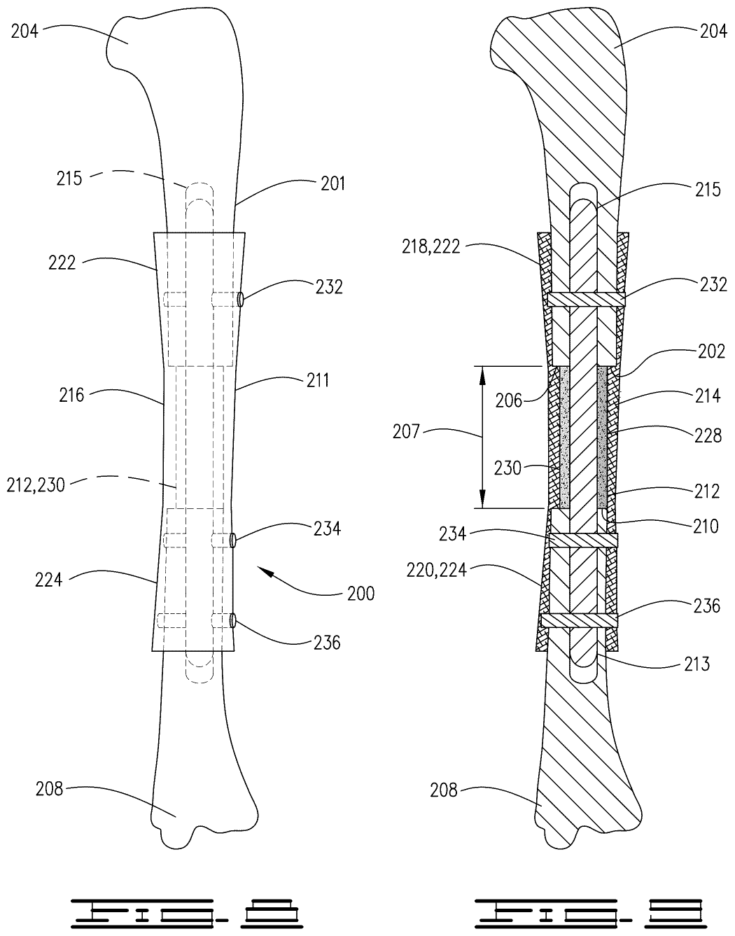

Repair apparatus 100 in FIGS. 6 and 7 is similar to that of FIG. 4. Repair apparatus 100 is shown with a critical bone defect 101. All of the components, including the scaffold 104 and the intramedullary nail 102, may be made from bioabsorbable and biodegradable PCL material. In the embodiment of FIG. 6, scaffold 104 may have a central portion 106 with first, or proximate and second, or distal ends 108 and 110 respectively disposed about the intramedullary nail 102. Scaffold 104 has a first, or proximate socket 112 at proximate end 108 and a second, or distal socket 114 at distal end 110. Socket 112 will extend over the end 116 of a first bone fragment 118 of critical bone defect 101. Socket 112 will envelop or encapsulate end 116. Socket 114 will extend over the end 120 of a second bone fragment 122 of critical bone defect 101. Sockets 112 and 114 are patient specific, in that the shape are designed specifically for the bone fragments of the critical bone defect to be repaired. One method of manufacturing the components, both the intramedullary nail 102 and the scaffold 104, is by using a 3D printer. The intramedullary nail and the scaffold may both be PCL. The scaffold may also be PCL-HA. The method of repair may comprise 3D printing the scaffold 104 and the intramedullary nail 102. In such a case, the intramedullary nail may be inserted through the scaffold 104, and the intramedullary nail 102 flexed to insert the ends thereof into the spaced apart ends of the first and second bone fragments 118 and 122 and into axial openings 128 in the first and second bone fragments 118 and 120. As a result, the openings 128 into which intramedullary nail 102 is inserted will not extend all the way through either of bone fragments 118 and 122.

At least one screw may be used above and below the critical defect to secure the scaffold 104. In the described embodiment, a screw 130 may extend through socket 112 of scaffold 104 into intramedullary nail 102 through bone fragment 118. A screw 132 may extend through socket 114 of scaffold 104 into intramedullary nail 102 through bone fragment 122. An additional screw 134 may be positioned below screw 132 and may extend through socket 114 of scaffold 104 into intramedullary nail 102 through bone fragment 122.

The embodiments described herein may use a metallic intramedullary nail or a non-metallic intramedullary nail. Metallic intramedullary nails may have slots just above and below the critical size bone defect to lock the intramedullary nail and the scaffold with the native bone by screws as described herein. Bioabsorbable non-metallic intramedullary nails may be locked in with screws as well, but no slots will be included in the bioabsorbable non-metallic intramedullary nails. Metal intramedullary nails may be micro-grooved and coated using, for example, the method described in U.S. Pat. No. 9,809,906. The metal intramedullary nail may be micro-grooved, soaked with collagen, and nanofibers, for example PCL nanofibers, may be placed in the grooves prior to the time the scaffold is placed. The nanofibers may be, for example, electrospun PCL nanofibers.

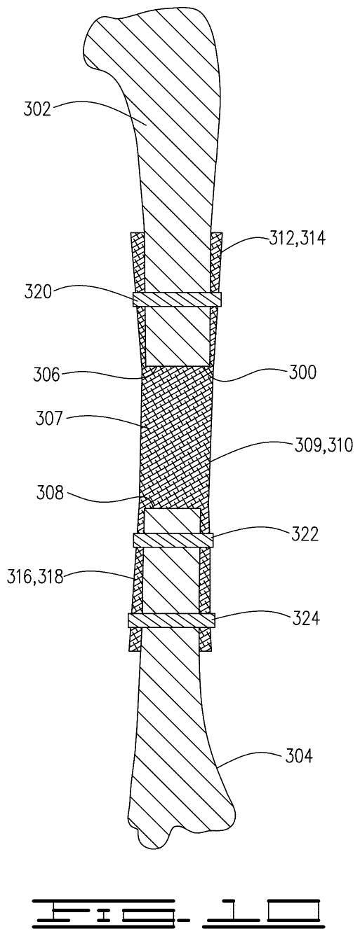

Another embodiment of a repair apparatus 200 is shown in FIGS. 8 and 9. In FIGS. 8 and 9, tibia 201 with a bone defect 202 is shown. Bone defect 201 comprises bone fragments 204 and 208 with a space 207 therebetween. First, or upper bone fragment 204 has an end 206 and second, or lower bone fragment 208 has an end 210. FIGS. 8 and 9 show repair apparatus 200 connecting bone fragments 204 and 208. Repair apparatus 200 may comprise a scaffold 211 disposed about an intramedullary nail 213 that extends between and into axial openings 215 in bone fragments 204 and 208. Scaffold 211 includes an inner portion or inner scaffold portion 212 and an outer scaffold portion 214. Inner and outer scaffold portions 212 and 214 may be referred to as first and second scaffold portions 212 and 214, respectively. In the embodiment of FIG. 8, inner scaffold portion 212 may be a 3D printed tissue-engineered critical-size bone scaffold, specific to the lesion size, site, and geometry. The outer scaffold portion 214 may be a 3D printed PCL or PCL-HA scaffold as described herein.

Outer scaffold portion 214 may have a central portion 216 disposed about the intramedullary nail 213, and first, proximate and second or distal ends 218 and 220, respectively. Outer scaffold portion 214 has a first, or proximate socket 222 at proximate end 218 and a second, or distal socket 224 at distal end 220. First socket 222 will extend over the end 206 of bone fragment 204, and will envelop, or encapsulate end 206. Second socket 224 will extend over the end 210 of bone fragment 208, and will envelop, or encapsulate end 210. Sockets 222 and 224 are patient specific, in that the shape are designed specifically for the bone fragments of the critical bone defect to be repaired.

Central portion 216 is hollow and thus central portion 216 and intramedullary nail 213 define an annular space 228 therebetween. Intramedullary nail 213 in one embodiment may be a PCL 3D printed bioabsorbable nail as described herein. As a result, axial opening 215 will not extend completely through either of bone fragments 204 and 208. Inner scaffold portion 212 may comprise a nanofiber mesh 230 circumferentially disposed about a portion of intramedullary nail 213 in annular space 228. Electrospun nanofiber mesh 230 may be produced by methods known in the art including those described herein. The nanofiber mesh 230 can be immobilized with bio molecules such as, for example, bone growth protein, peptide, and growth factors. The nanofiber mesh serves as a carrier for BMSC, bone growth factors, antimicrobial agents, and heal-tracking molecules, promoting natural bone regeneration, resisting infection and monitoring healing.

At least one screw may be used above and below the critical defect to secure the scaffold 211. In the described embodiment, a screw 232 may extend through socket 222 of scaffold 211 into intramedullary nail 213 through bone fragment 204. A screw 234 may extend through socket 224 of scaffold 211 into intramedullary nail 213 through bone fragment 208. An additional screw 236 may be positioned below screw 234 and may extend through socket 224 of scaffold 211 into intramedullary nail 213 through bone fragment 208.

A method of repairing may comprise, for example, 3D printing the intramedullary nail 213 and depositing the electrospun nanofiber mesh 230 onto the intramedullary nail 213. The outer scaffold portion 214 can then be 3D printed around the nanofiber mesh 230. Because the components are flexible, the ends of intramedullary nail 213 can be flexed and inserted into openings in the ends 206 and 210 of bone fragments 204 and 208. First and second sockets 222 and 224 will be placed over the ends 206 and 210 of bone fragments 204 and 208, respectively.

Each of the embodiments described herein have axial openings in the bone fragments into which the intramedullary nails are inserted. In some cases, the axial opening extends from a bottom end of the lower bone fragment through the lower bone fragment and into the upper bone fragment. This will be the case where a metal, non-flexible intramedullary nail is used. When flexible intramedullary nails are used, the ends thereof may be inserted into axial openings at the spaced apart ends of the bone fragments, and the axial opening may extend only part way into the spaced apart bone fragments.

In an additional embodiment, a bone defect 300 with first and second bone fragments 302 and 304 having spaced apart ends 306 and 308 may be repaired with a repair apparatus 307. Repair apparatus 307 comprises a scaffold 309 with a central portion 310 positioned between spaced apart ends 306 and 308 of first and second bone fragments 302 and 304. Scaffold 309 has a first socket 312 at a first end 314 thereof and a second socket 316 at a second end 318 thereof. First and second sockets 312 and 316 extend over and envelop ends 306 and 308 of first and second bone fragments 302 and 304, respectively.

Scaffold 309 may be made from a biocompatible material and may in one example be 3D printed. In one embodiment, the scaffold may be a 3D printed PCL scaffold as previously described herein. In the embodiment of FIG. 10, no intramedullary nail is used. At least one screw may be used above and below bone defect 300 to secure scaffold 309. A screw 320 may extend through socket 312 of scaffold 309 into first bone fragment 302. A screw 322 may extend through socket 316 of scaffold 309 into second bone fragment 304. An additional screw 324 may be positioned below screw 322 and may extend through socket 316 of scaffold 309 into second bone fragment 304.

The method and apparatus of the current disclosure were used on a rabbit tibia. For the critical bone defect repair surgery, an orthopedic oscillating bone created a 20 mm defect (same as scaffold length) in the rabbit tibia inferior to where the fibula fuses with the tibia. This defect length represents 20% of the tibia's length, which has been used by others as a measure of a critical-sized defect. Prior to insertion in the tibia, a series of drill bits were used to make a hole along the medullary cavity from the cut end of the tibia. The drilling resulted in a through hole at the proximal end and a blind-ended hole at the distal end for press-fitting the intramedullary nail when it is pushed from the proximal end of the drilled hole. The length of the proximal-to-distal hole including the bone scaffold was the same as the intramedullary nail length. The bone scaffold was placed in the bone defect. The scaffold was secured by pushing it to the distal and proximal ends to encapsulate the ends of the proximal and distal bone fragments with the scaffold extruded ends. The intramedullary nail was then inserted into the medullary cavity through the bone scaffold. Three side holes were drilled through the slots of intramedullary nail for the self-locking screws to fasten the intramedullary nail with bone and scaffold. For absorbable IM nail, three side holes will be drilled through bone and IM nails to secure them by the self-locking screws. The process was generally as follows:
1. Cut skin and subcutaneous tissue layers to see the mid-physics of the tibia bone.
2. Create a 20 mm defect where the fibula fuses with the tibia. A 20 mm bone scaffold will be placed along the tibia as a measurement guide to cut the bone. A pulsating saw was used to cut the tibia bone of a similar size to guide the scaffold.
3. The novel PCL-HA scaffold has a 20 mm porous structure with extruded hollow ends. The extruded ends of the bone scaffold were designed to fit into the proximal and distal bone ends.
4. PCL IM nail was inserted into the bone scaffold and installed to by pushing it to the distal and proximal ends of cut bone ends.
5. Three through holes were created laterally, two at the proximal end and one at the distal ends of the bone scaffold. The scaffold is secured with the bone and IM nail with two metal screws. The intramedullary nail is secured with bone by the third metal screw.
6. Subcutaneous tissue layers and skin was closed with vicryl 4/0 suture thread and 3/0 silk sutures, respectively.

The described method provides for the repair of critical bone defects without the need for an external jig. Polycaprolactone-hydroxyapatite (PCL-HA) scaffolds and PCL intramedullary nails were successfully developed, solving one of the most challenging aspects of delivering the scaffolds and intramedullary nails for the rabbit tibia using a surgical approach. In addition, PCL-HA and PCL are biocompatible and degradable. Significant attachment of BMSCs on the scaffold surface have been observed. Bioabsorbable scaffold and intramedullary nails that can be absorbable, may offer many advantages over the autologous bone substitute solutions, including reduced recovery time, load bearing during the recovery process, and no need for future surgical procedures to remove the implants due to their bioabsorbable property. In each of the described embodiments, the intramedullary nail may be metallic or non-metallic and may be made from a bioabsorbable material.

In each of the described embodiments, the scaffold and/or the nanofiber mesh acts as a bone graft, and because it is comprised of bioabsorbable material, the scaffolds and/or nanofiber mesh will be absorbed into the body of the patient and replaced by bone as the spaced apart fragments grow together.

What is claimed is:

1. A repair apparatus for repairing bone defects consisting of spaced apart first and second bone fragments of a bone, the repair apparatus comprising:
a bioabsorbable scaffold having first and second ends spanning the space between the spaced apart bone fragments, wherein the first end of the scaffold envelops an end of the first bone fragment and the second end of the scaffold envelops an end of the second bone fragment;
a first screw fixing the scaffold to the first bone segment; and
a second screw fixing the nail to the second bone segment.

2. The repair apparatus of claim 1, further comprising an intramedullary nail extending through the scaffold into the first and second bone segments, wherein the first screw fixes the scaffold and the intramedullary nail to the first bone segment, and wherein the second screw fixes the scaffold and the intramedullary nail to the second bone segment.

3. The repair apparatus of claim 2, further comprising a nanofiber mesh surrounding at least a portion of the scaffold.

4. The repair apparatus of claim 2, the intramedullary nail comprising a metal nail.

5. The repair apparatus of claim 2, the intramedullary nail comprising a bioabsorbable nail.

6. The repair apparatus of claim 2, the bioabsorbable scaffold and the intramedullary nail defining an annular space therebetween, the repair apparatus further comprising a nanofiber mesh disposed about the intramedullary nail in the annular space.

7. A method of repairing a bone defect comprising:
manufacturing a scaffold with first and second sockets at first and second ends thereof;
positioning the scaffold in a space between first and second bone fragments of the critical bone defect;
inserting an end of the first bone fragment into the first socket defined at a first end of the scaffold;
inserting an end of the second bone fragment into the second socket defined at a second end of the scaffold; and
pressing an intramedullary nail through the scaffold and into axial cavities in the first and second bone fragments.

8. The method of claim 7 further comprising, affixing the scaffold and the intramedullary nail to the first and second bone fragments with screws.

9. The method of claim 7, wherein the scaffold comprises an inner scaffold portion disposed about the intramedullary nail and an outer scaffold portion disposed about the inner scaffold portion, one of the inner and outer scaffold portions comprising a nanofiber mesh scaffold.

10. The method of claim 9, the inner scaffold portion comprising a nanofiber mesh scaffold and the outer portion comprising a 3D-printed PCL scaffold.

11. The method of claim 9, the inner scaffold portion comprising a 3D-printed PCL scaffold and the outer portion comprising a nanofiber mesh.

12. The method of claim 7, further comprising;
fixing the first socket to the first bone fragment with a fastener that extends through the first socket and the intramedullary nail; and
fixing the second socket to the second bone fragment with a fastener that extends through the second socket and the intramedullary nail.

13. The method of claim 7, wherein the intramedullary nail is a flexible non-metallic intramedullary nail.

14. The method of claim 13, wherein the intramedullary nail is a PCL intramedullary nail.

15. A repair apparatus for repairing bone defects consisting of spaced apart first and second bone fragments of a bone, the repair apparatus comprising;
a scaffold extending from an end of the first bone segment to an end of the second bone fragment, the scaffold having a first socket at a first end thereof extending over and enveloping a portion of the first bone fragment and having a second socket at a second end thereof extending over and enveloping a portion of the second bone fragment, and;
a fastener extending through at least one of the first and second sockets into the corresponding first or second bone fragment.

16. The repair apparatus of claim 15, further comprising an intramedullary nail spanning a space between the first and second bone fragments and extending into the first and second bone fragments, wherein the at least one fastener extends through the intramedullary nail.

17. The repair apparatus of claim 16, wherein the intramedullary nail is a flexible, non-metallic intramedullary nail.

18. The repair apparatus of claim 16, the scaffold further comprising:
an inner scaffold portion disposed about the intramedullary nail; and
an outer scaffold portion disposed about the inner scaffold portion, wherein one of the inner and outer scaffold portions is comprised of nanofiber mesh.

19. The repair apparatus of claim 18, wherein the inner scaffold portion comprises nanofiber mesh, and wherein the first and second sockets are on the outer scaffold portion.

20. The repair apparatus of claim 16, wherein the intramedullary nail is a 3D-printed PCL intramedullary nail.
